(19) United States
(12) Patent Application Publication
(10) Pub. No.: US 2017/0074695 A1
(43) Pub. Date: Mar. 16, 2017

(54) Sensor Block, Pipe, and Production Method

(71) Applicant: seleon GmbH, Heilbronn (DE)

(72) Inventor: Martin Baecke, Dessau (DE)

(73) Assignee: seleon GmbH, Heilbronn (DE)

(21) Appl. No.: 15/123,507

(22) PCT Filed: Mar. 3, 2015
(86) PCT No.: PCT/EP2015/054374

§ 371 Date: Sep. 2, 2016

(30) Foreign Application Priority Data
Apr. 3, 2014 (EP) 14157563.9

(51) Int. Cl. G01F 1/46; A61M 16/08; G01F 15/18

(52) U.S. Cl. CPC G01F 1/46; G01F 15/185; A61M 16/085; A61M 16/0858; A61M 2207/00

(57) ABSTRACT

The embodiments of the present invention relates to a sensor block for measuring fluid flow or pressure in a tube. The sensor block comprises a sensor and a housing. The housing has a resilient clamp part which is shaped such that it can be plugged onto a tube along the radial direction and is part of the tube in the plugged state. The invention also relates to a tube, which is produced by means of MID technology. The tube comprises conductor paths and a sensor, which is firmly connected to the tube. The tube further comprises two elongated grooves for releasable attachment of a sensor block. The embodiments of the present invention further relates to a production method, in which both a sensor block and a tube are manufactured with an identical mask set, injection molding tool or control program component.

The invention relates to ventilation equipment, in particular, the sensor components thereof.

The invention relates to sensor blocks according to the preamble of claim 1, a tube with a sensor, tubes according to the preambles of the claims 12 to 14 as well as a production method.

According to Wikipedia, a microsystem is a miniaturized device, a module or unit, whose components have the smallest dimensions in the micrometer range and interact as a system. Typically, a microsystem comprises one or more sensors, actuators, and control electronics on a substrate or chip. Microsystems technology is the study of the development of microsystems and their implementation techniques.

In English, the abbreviations MEMS and MOEMS, which stand for micro-electro-mechanical systems and micro-opto-electro-mechanical systems are common. However in Asia, mainly Japanese, publications also include the advanced designation micromachines. The use of microsystems is conceivable and reasonable where sensors, actuators, and electronics interact with one another. Medical devices and products in the fields of security technology, sports, life sciences and logistics can be made versatile, simpler, smarter, smaller and more powerful with the help of microsystems.

The production of both pressure and flow sensors by means of microsystems technology is well known. Differential pressure sensors SDP1108 and SDP2108 from SENSIRON AG may be mentioned as examples. As these sensors have a thermal sensor element, they are, strictly speaking, flow sensors, which measure the low gas flow through a capillary. As part of this application, differential pressure sensors according to the data sheet of SDP1108 and SDP2108 also refer to flow sensors, particularly thermal mass flow sensors, which measure the pressure drop across a capillary.

Molded Interconnect Devices, in short MID, are electronic components, in which metallic conductor paths are applied to injection molded plastic carriers. Main areas of application for the MID technology are the automotive, industrial automation, medical technology, household appliances, telecommunications equipment, the measurement and analysis technology and aerospace.

The improved freedom of design and integration of electrical and mechanical functions in a molded part can lead to miniaturization of the module. Rationalization potential is in reducing the number of parts by saving material and shortening the process chain. Reliability can also be increased by reducing the number of assembly steps.

By the use of MID technology, the conventional material mix of a combination of a printed circuit board and mechanical component, which usually consists of a variety of materials, can be replaced by a metallized plastic part (MID). MIDs are made from recyclable thermoplastics and are less critical than conventional printed circuit boards when it comes to disposal. However, the base material for a printed circuit board is generally a non-recyclable thermosetting plastic that is difficult to dispose of

Devices for performing the CPAP (continuous positive airway pressure) therapy are also known, see WO 2004/045693 A2, with further references. A CPAP device applies a positive overpressure of up to approx. 30 mbar in the respiratory tract of the patient via a hose and a nose mask by means of a compressor, preferably a humidifier. This overpressure is to ensure that the upper respiratory tract remains fully open during sleep during the whole night, so that no obstructive breathing disorders (apnea) occur (DE 198 49 571 A1).

Sterile filters are also commercially available. They are made of, for example a porous plastic body made of polypropylene or Teflon (PTFE, polytetrafluoroethylene) to trap microorganisms. Typical bacteria have a size from 0.2 to about 4 microns. Viruses are smaller than 0.3 micron and bacteriophages can have a size of up to 0.01 micron.

U.S. Pat. No. 7,814,907 B2 discloses a system for applying gases for ventilators. A blower forces air through a humidification chamber and an artificial respiratory duct to a patient.

Approximately one quarter of the length of the artificial respiratory duct from the humidification chamber, the breathing conduit is interrupted by, for example a T-shaped tube having a connector for a sensor housing. The sensor in the sensor housing can measure humidity, temperature and/or flow rate. A filter, for example, a SYMPATEX® film is provided between the interior of the artificial respiratory duct, in particular, the interior of the tube, and the sensor. The filter is to keep dust, vapor, bacteria and viruses away from the sensor. The filter can be glued to the tube. The tube and the sensor housing can be connected by a thread, a snap fastener, a bayonet fastener or a silicone seal. The sensor housing itself may be located in the tube and consist of a tube, whose ends are closed by filter.

The object of the invention is to provide an improved sensor block, an improved tube and an improved production method.

This object is achieved by the theories of the independent claims.

Preferred embodiments of the invention are subject matter of the dependent claims.

An advantage of a sensor block having a housing with an elastic clamp part, which is shaped so that it can be plugged onto a tube in the radial direction and is a portion of the tube in the plugged condition, is that the sensor block can be easily mounted on the tube. This is advantageous during assembly of, e.g. CPAP devices. Further, it allows the use of tubes as disposable articles and reuse of the relatively expensive sensor block at the same time. To avoid contamination of the sensor, the openings of the tube are closed with sterile filters.

Elongated projections on the clamp parts, which extend parallel to the axis of the tube, and/or corresponding grooves in the tube allow reproducible positioning of the sensor block on the tube.

The use of a differential pressure sensor advantageously allows the use of pitot tubes or orifice plates for diagnostic measurement of the gas flow in the tube.

Unlike a simple pitot tube or a Prandtl pitot tube, a double pitot tube also allows diagnostic measurement of the gas flow regardless of the direction of flow in the tube.

A connector for a hose advantageously allows supply of other gases, in particular, oxygen, or taking samples from the breathing air.

A differential pressure sensor, which measures the pressure difference between the connector for the hose and the interior of the tube or around the tube advantageously allows flow measurement of the gas supplied or withdrawn from the connector. This flow measurement is more accurate when the connector is connected to the interior of the tube via a capillary opening.

The flow measurement in the tube may be more accurate if a double pitot tube is not used and the differential pressure is measured across an orifice plate (86) made of porous material. The reason for this is the flow-dependent flow profile in the tube.

A sensor block and a tube can be advantageously designed so that both of them can be produced with a single mask set by means of MID technology, at least partially identical injection molds and at least partially identical control program components.

Preferred embodiments of the invention are explained in detail in the following with reference to the accompanying drawings. Illustrated are:

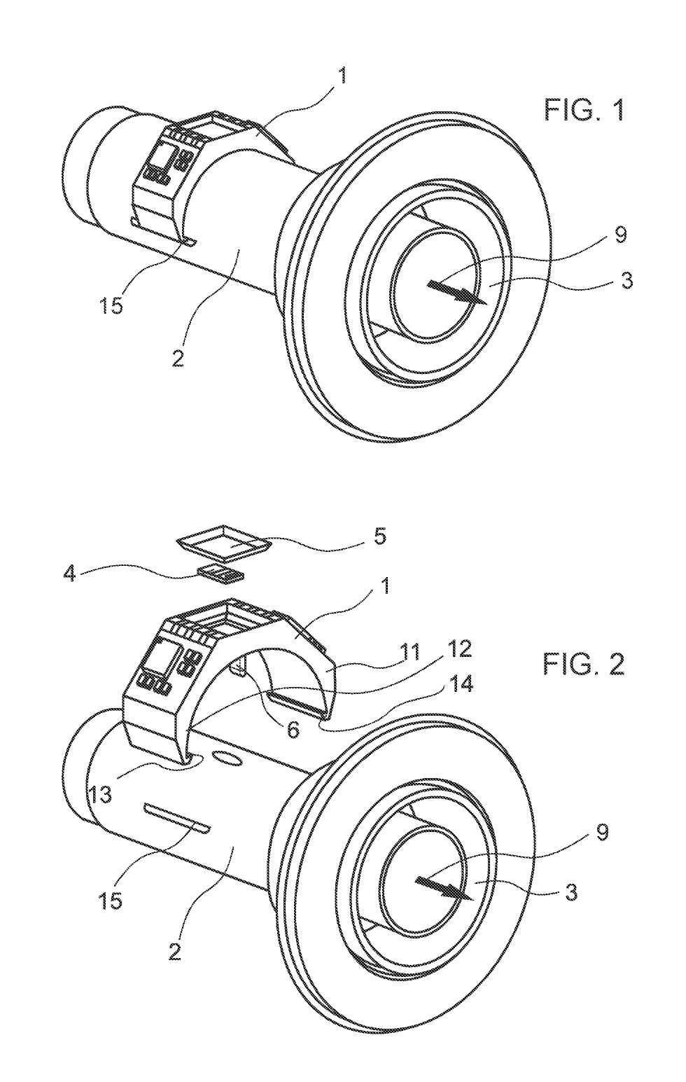

FIG. 1 shows a flow tube (of the present invention) of a CPAP device.

FIG. 2 is an exploded view of the flow tube shown in FIG. 1;

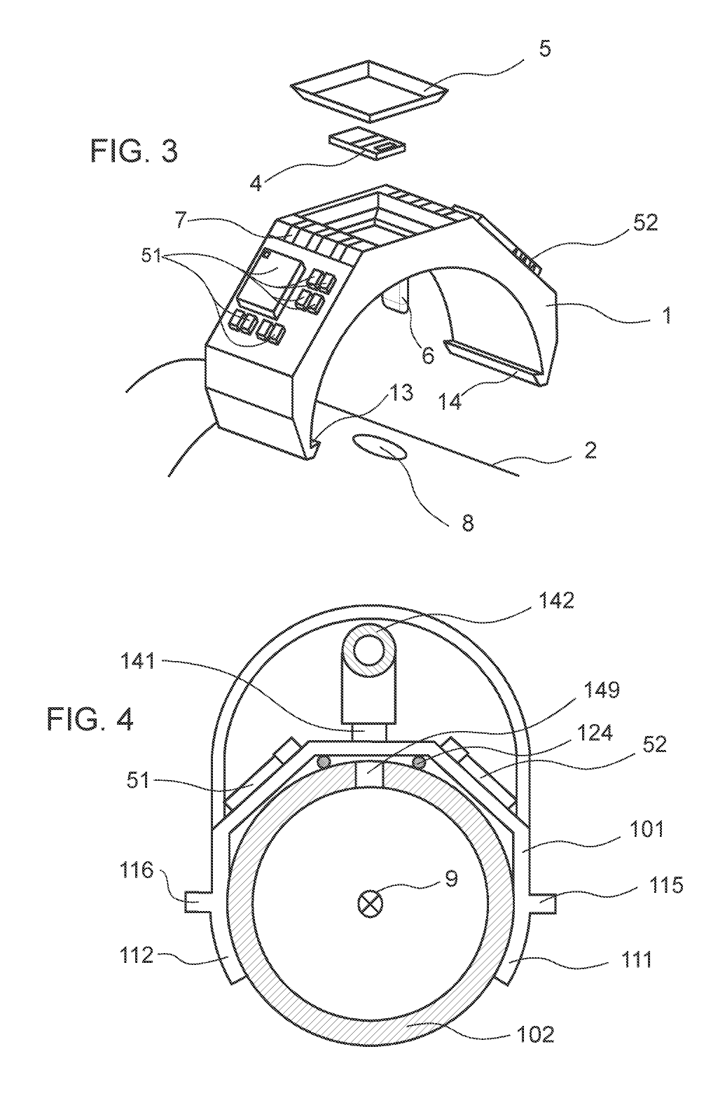

FIG. 3 is an exploded view of the sensor block of the present invention illustrated in FIGS. 1 and 2;

FIG. 4 shows a cross-section of a second embodiment of a sensor block and a flow tube of the present invention.

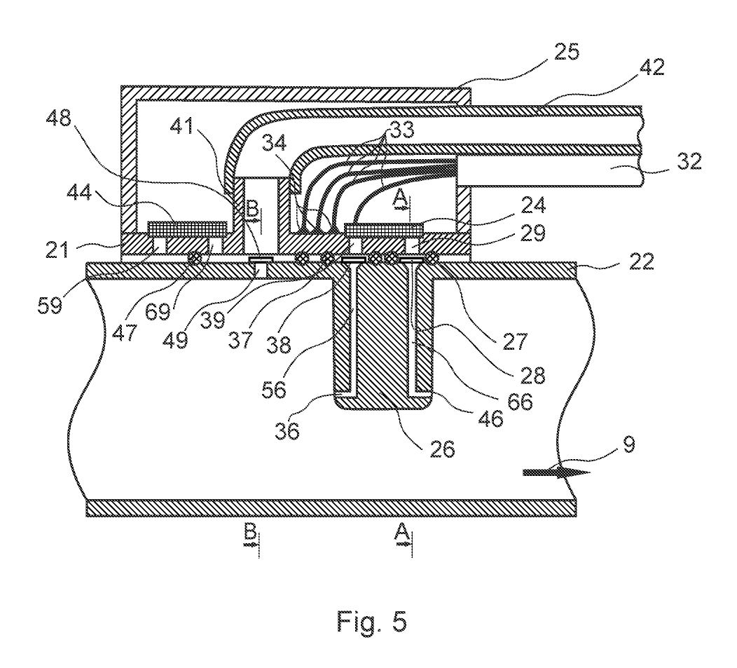

FIG. 5 shows a longitudinal section of a third embodiment of a sensor block and a flow tube of the present invention.

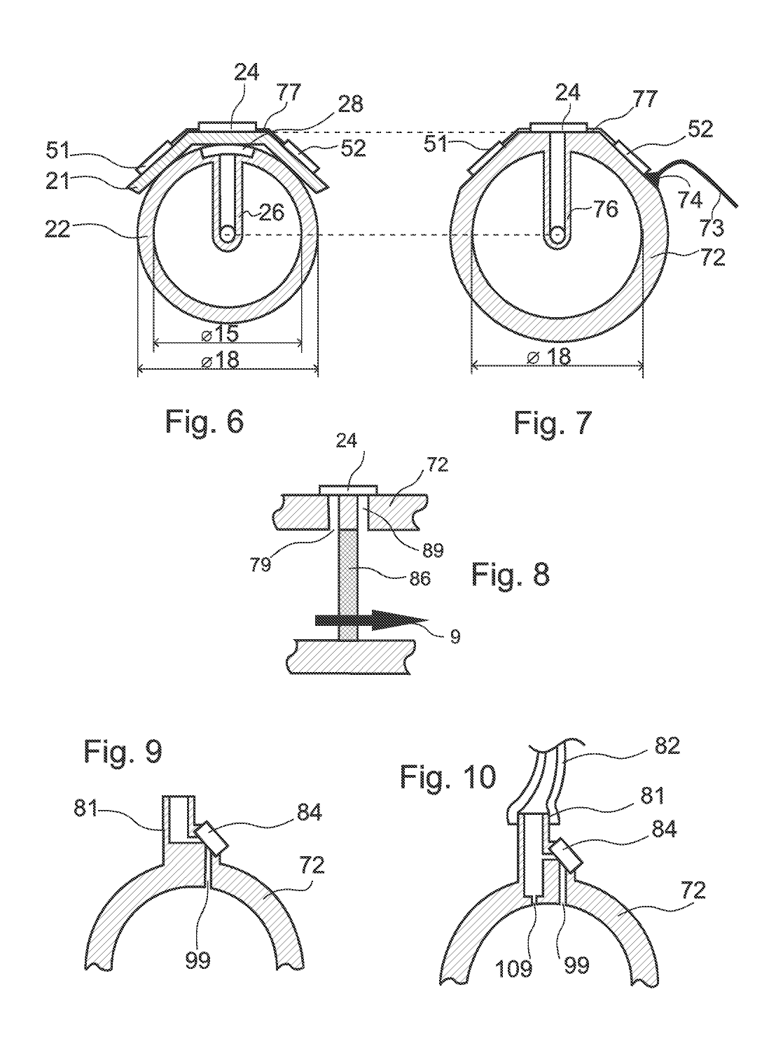

FIG. 6 shows a cross-section of the third embodiment of a sensor block of the present invention and a flow tube of the present invention along A-A line in FIG. 5;

FIG. 7 shows a cross-section of a fourth embodiment of a flow tube of the present invention;

FIG. 8 shows a longitudinal section of a fifth embodiment of a flow tube of the present invention;

FIG. 9 shows a cross-section of a sixth embodiment of a flow tube of the present invention; and

FIG. 10 shows a cross-section of a seventh embodiment of a flow tube of the present invention.

FIG. 1 shows a flow tube 2 (of the present invention) for a CPAP device. The front, expanded end of the flow tube 2 is mounted in a wall of the CPAP device. The artificial respiratory hose connector 3, which remains accessible from the outside after assembly of the CPAP device is also there. A sensor block 1 is mounted on top of the flow tube 2. The sensor block 1 may include sensors for flow and/or pressure measurement in the flow tube 2.

FIG. 2 shows the removed sensor block 1. FIG. 3 shows the crop of FIG. 2 with the sensor block 1. The sensor block 1 is C-shaped. The two ends are referred to as clamp parts 11 and 12. The sensor block 1 with its two clamp parts 11 and 12 comprises a little more than half or, in other words, slightly more than 180° of the flow tube 2. For a more reliable fixture, each clamp part 11, 12 has a longitudinal projection 13 and 14 at its end. Each longitudinal projection 13 or 14 engages in a groove 15 on the flow tube 2. In FIGS. 1 and 2, only the groove 15 is located on the side of the flow tube 2 facing the viewer.

A pitot tube 6 protrudes from below the sensor block. If the sensor block 1, as shown in FIG. 1, is mounted on the flow tube 2, the pitot tube 6 protrudes through the opening 8 into the interior of the flow tube 2. The structure of the pitot tube 6 is similar to the structure of the pitot tube 26 shown in FIG. 5 with two openings, wherein one opening is directed towards the direction of flow 9 and another opening opposite the direction of flow 9. In the case of the flow tube 2, the direction of flow 9 points to the artificial respiratory hose connector 3.

In the so-called single-duct ventilators as illustrated by, for example, CPAP devices, a specific leak, over which the exhaled carbon dioxide is washed out, is provided in or near the ventilation mask. A net flow results from the ventilator to the respiratory mask towards the direction of flow 9 averaged by a single act of breathing. Nevertheless, air can be pushed back against the direction of flow 9 to the CPAP device when exhaling air.

The flow tube 2 and/or the sensor block 1 are made of an elastic material so that the sensor block 1 can be repeatedly plugged and removed from the flow tube 2. The sensor block 1 is designed in MID technology. On a plastic body, conductor paths 7, for example made of copper, and electronic components are connected to the conductor paths. For example, FIG. 3 shows the components of a preamplifier 51 and another electrical component 52. Attachment is, e.g. by soldering, in particular, flow soldering or bonding, for example with silver conductive adhesive. This type of attachment is of a mechanical nature and provides an electrical contact between the electronic components and the conductor paths 7.

In particular, a differential pressure sensor 4 and a cover 5 are mounted on the sensor block 1. As mentioned above, differential pressure sensors also refer to flow sensors within the scope of this document, in particular, thermal mass flow sensors, which measure the pressure drop across a capillary as the sensors SDP1108 and SDP2108.

In order to mechanically stress the conductor paths 7 and the electronic components in the central part of the sensor block 1 as little as possible, the central part of the sensor block 1 is designed more rigid than the clamp parts 11 and 12. This can be achieved by designing the central part thicker and/or making it from a stiffer material than the clamp parts 11, 12. If the clamp parts 11, 12 cover more than 180° of the flow tube 2, the grooves 15 and the projections 13, 14 are not required. Correct positioning of the sensor block 1 relative to the flow tube 2 can also be ensured in this case by the pitot tube 6 and the opening 8. If the projections 14 and 15 and the grooves 15 are present, the sensor block may also cover less than 180° of the flow tube 2. If the projections 13 and 14 have a hook-shaped cross-section, and the grooves 15 are accordingly shaped, it is sufficient if the sensor block 1 covers only a small angular range of the flow tube 2, namely 60°, 30° or less.

FIG. 4 shows a cross-section of a second embodiment of a sensor block 101 of the present invention and a flow tube 102 of the present invention with preamplifier 51 and electrical component 52. The direction of flow 9 extends into the drawing plane. The sensor block 101 has on its outside ribs 115 and 116 to facilitate the removal and attachment of the sensor block 101 onto the flow tube 102. In this embodiment, the clamp parts 111 and 112 do not have any protrusion, and the flow tube 102 also does not have any groove. The sensor block 101 covers about 240° of the flow tube 102. The sensor block 101 has a hose connector 141 for a hose 142. The flow in the tube 142 is measured by means of a flow sensor. How the flow measurement is carried out is explained on the basis of the sections shown in the FIGS. 9 and 10. The hose 142 may be used to supply oxygen or other gases or mixtures of gases for diagnostic or therapeutic purposes. The hose 142 can also be used for taking samples especially from exhaled gas. In the flow tube 102, an opening 149, which is pneumatically connected to the hose connector 141, is located below the hose connector 141. The sealing ring 124 prevents pneumatic connection to the surrounding area. For illustration purposes, FIG. 4 again shows the components of a preamplifier 51 and another electrical component 52.

The sensor block 101 shown in FIG. 4 can be supplemented by a second differential pressure sensor 4 and a pitot tube 6, as shown in FIG. 5. Conversely, the sensor block 1 can also be supplemented by a second differential pressure sensor and a hose connector 141, as shown in FIG. 5.

FIG. 5 shows a longitudinal section of a third embodiment of a sensor block of the present invention with an MID component 21 and a cover 25 as well as a flow tube 22 of the present invention. FIG. 5 essentially illustrates a combination of the sensor blocks 1 and 101, shown in FIGS. 1-3 and in FIG. 4. In contrast to such a combination, the gas chamber is separated in the flow tube 22 is separated from the gas chamber in the sensor block 1 by the sterile filters 28, 38 and 48.

If the flow tube 22 shown in FIG. 5 is used near a respiratory mask, both inhaled and exhaled air flows through the flow tube 22. The direction of flow 9 can therefore be arbitrarily selected, parallel to the axis of the flow tube 22 and positive and negative gas flows occur independent of the choice, namely gas flows along the direction of flow 9 or opposite the direction of flow 9. In terms of quantity, the positive and negative gas flows are approximately the same.

In order to measure the gas flows along and opposite the direction of flow 9, the double pitot tube 26 is symmetrical to a plane perpendicular to the direction of flow 9. In particular, an opening 46 points to the direction of flow and an opening 36 opposite the direction of flow. The pressure at the opening 36 is applied to an input of the differential pressure sensor 24 via a channel 56 through an opening 39. The channel 56 is pneumatically connected to the opening 39 via a sterile filter 38, wherein the sealing ring 37 provides hermetic sealing. Similarly, the pressure in the opening 46 is applied to the other input of the differential pressure sensor 24 via the channel 66 through the opening 29. The channels 56 and 66 widen directly below the sterile filters 28 and 38, whose surface is designed as large as possible as a sterile filter shows a certain flow resistance for small air molecules such as N₂, O₂ and H₂O. Thus, the flow resistance is kept low by the sterile filter. The channel 66 is pneumatically connected to the opening 29 via a sterile filter 28 and hermetically sealed by a sealing ring 27. It is known to a person skilled in the art that the flow rate can be calculated using the Bernoulli equation, on the basis of the signal returned by the differential pressure sensor 24. The Bernoulli equation is parameterized, namely adapted to the specific measurement case. The diagnosis of the gas flow results from the diagnosis of the signal returned by the differential pressure sensor 24.

The MID component 21 also has a hose connector 41 for a hose 42. For example, oxygen can be forced in this way from the tube 42 into the flow tube 22, via another sterile filter 48 and a capillary 49. Another differential pressure sensor 44 is provided to measure the pressure difference across the capillary opening 49. The first pressure port of the differential pressure sensor is pneumatically connected to the interior of the hose connector 41 via the opening 69 and hermetically sealed by the sealing ring 47. The differential pressure sensor 44 does not exactly measure the differential pressure at the capillary opening 49 as the second pressure port of the differential pressure sensor 44 is connected with the surroundings and not to the interior of the flow tube 22 via the opening 59. However, the pressure in the flow tube 22 usually deviates from the ambient pressure only by a few 10 mbar. If the pressure difference measured by the differential pressure sensor 44 is large by some 10 mbar, the difference between the ambient pressure and the pressure in the flow tube 22 may be neglected. However, the differential pressure between the interior of the flow tube 22 and the surrounding area is usually known, so that the pressure measured by differential pressure sensor 44 can be corrected by this pressure difference.

Likewise the channels 56 and 66, the capillary 49 may be expanded below the sterile filter 48. This may be particularly advantageous while taking gas samples via the tube 42. If oxygen is supplied through the hose 42, the oxygen is typically under sufficiently high pressure so that the oxygen flow is sufficient despite the differential pressure between the capillary opening 49 and the sterile filter 48. Under this condition, a high differential pressure is more advantageous because a high differential pressure can be more accurately measured by the differential pressure sensor 44. Therefore, FIG. 5 does not show any expansion of the capillary opening 49 below the sterile filter 48.

In another embodiment, another opening may be provided below the opening 59 in the flow tube 22. Both openings are pneumatically connected in this embodiment by a sealing ring.

Instead of the sealing rings 27, 37 and 47, a single seal that is made of, e.g. a soft material, and must have three openings separated by the sealing material for the application in FIG. 5 may be used.

The MID-component 21 is manufactured in MID technology. Therefore, by way of example, the solder joints 34 and the conductor 33 of the electrical lead 32 are shown, over which the differential pressure sensors 24 and 44 are supplied with electric power and are read out. A cover 25 protects the components on the MID component 21.

FIG. 6 shows a cross-section along the line A-A in FIG. 5 with the MID-component 21, the flow tube 22, the double pitot tube 26, the sterile filter 28, the differential pressure sensor 24, preamplifier 51 and an electronic component 52.

FIG. 7 shows a flow tube 72, which is manufactured in MID technology and includes the double pitot tube 76. The MID component 21 illustrated in FIGS. 5 and 6 is part of the flow tube 72 in the embodiment shown in FIG. 7. On top of the flow tube 72, preamplifier 51, differential pressure sensor 24 and an electronic component 52, conductor paths 77, solder joints 74 and an electrical connector 73 are shown again. The advantage of the embodiment shown in FIG. 7 is that the dimensions of the flow tube 72 are equal to the MID-component 21 at least in the upper area, so that the same mask set, one or more identical injection molds and/or the control program that is identical in one or more components can be used for the production of the MID-component 21 same as for the production of the flow tube 72. Mask set and injection molding tools can be summarized under the broader term of tool set.

FIG. 8 shows a longitudinal section of a fifth embodiment of a flow tube 72 of the present invention. In this embodiment, a pressure difference across a porous orifice plate 86 is produced in a known manner by a gas flow in a flow tube 72. The direction of flow 9 is set from left to right. The pressure difference is applied to the two ports of a differential pressure sensor 24 via two openings 79 and 89. In this embodiment, the flow tube 72 is designed in MID technology. Nevertheless, the person skilled in the art will recognize that the double pitot tube 26 can also be replaced by a porous orifice plate 86 in the embodiment illustrated in FIG. 5.

The FIGS. 9 and 10 illustrate alternative embodiments for the left part of the flow tube 22 shown in FIG. 5 along line B-B. In both cases, the flow tube 72 is designed in MID technology as shown in FIGS. 7 and 8. In FIG. 9, no hose is attached to the hose connector 81. One port of the differential pressure sensor 84 is pneumatically connected with the surrounding area via the hose connector 81. The other port of the differential pressure sensor 84 is connected with the interior of the flow tube 72 via the opening 99. Thus, the differential pressure sensor 84 measures the pressure difference between the inside of the flow tube 72 and the surrounding area.

In contrast, in FIG. 10, a tube 82 is plugged onto the hose connector 81 and a capillary opening 109 is provided as a bypass to the differential pressure sensor 84. Thus, the hose 82 allows supply of additional gases, in particular oxygen to the flow tube 72 or taking of gas samples from the flow tube 72. The differential pressure at the capillary 109 is used to determine the gas flow from the tube 82 into the flow tube 72 and vice versa.

The flow tubes 72 illustrated in FIGS. 9 and 10 can be produced with very similar injection molds, which differ only by a different punch. The punch for the production of the flow tube 72 shown in FIG. 9 is a little shorter as the cavity in the hose connector 81 is also slightly shorter. Conversely, the punch for the production of the flow tube 72 shown in FIG. 10 is slightly longer, so as to also produce the capillary 109.

If the differential pressure sensor 84 in FIG. 9 comprises a thermal sensor element, strictly speaking, is a flow sensor, the embodiment illustrated in FIG. 9 may also include a tube 82 that is plugged onto the hose connector 81, such that either gases may be supplied to the interior of the flow tube 72 or gas samples may be taken from the interior of the flow tube 72.

Although the invention has been described in connection with gas so far, it is clear to those skilled in the art that the flow measurement also works with liquids and therefore, the term gas can be replaced by the broader term fluid.

The invention has been explained in detail, based on preferred embodiments. However, it is obvious to a person skilled in the art that various changes and modifications can be made without departing from the spirit of the invention. Therefore, the scope is defined by the following claims and their equivalents.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A sensor block for measuring fluid flow or pressure in a tube, the sensor block comprising:
a sensor; and
a housing with a resilient clamp part for the sensor block to be removably mounted on the tube;
wherein the resilient clamp part is shaped such that the resilient clamp part can be plugged onto the tube along the radial direction and the resilient clamp part radially covers part of a wall of the tube.

17. The sensor block of claim 16, wherein the resilient clamp part has a longitudinal projection, which extends parallel to a longitudinal axis of the tube when the sensor block is mounted on the tube.

18. The sensor block of claim 16, wherein the sensor is a differential pressure sensor and the sensor block has two openings facing the tube; each opening is pneumatically connected to a pneumatic connection of the differential pressure sensor.

19. The sensor block of claim 16, wherein the sensor is a differential pressure sensor;
wherein the housing further comprises a double pitot tube, which has two openings in opposite directions and two channels, with each channel pneumatically connects each opening of the double pitot tube to the differential pressure sensor; and
wherein the double pitot tube is adapted to protrude into the tube when the sensor block is mounted on the tube.

20. The sensor block of claim 16, wherein the sensor block is adapted to be mounted on the tube that further comprises a port for a hose to be connected and the sensor is a pressure sensor that is pneumatically connected to the port so that the pressure sensor can measure pressure in the port.

21. The sensor block of claim 16, wherein sensor block is adapted to be mounted on the tube that further comprises an orifice plate mounted across an interior of the tube; the orifice plate being made of porous material; the tube has a first opening on a side upstream of the orifice plate and a second opening on a side downstream of the orifice plate in relative to a direction of flow;
wherein the sensor is a differential pressure sensor having a first port pneumatically connected to the first opening of the tube and a second port pneumatically connected to the second opening of the tube.

22. A method of producing the sensor block of claim 16 and the tube, comprising the steps of:
using a mask set, a tool set, an injection mold and/or a control program component to produce the sensor block;
using the mask set, the injection mold, and/or the control program component to produce the tube.

23. A sensor system for measuring fluid flow or pressure, the system comprising:
a tube, conductor paths, and a sensor which is mounted to the tube; wherein the tube is produced by means of MID technology.

24. The system of claim 23,
wherein the sensor is a differential pressure sensor;
wherein the tube has a double pitot tube protruding into the tube; the double pitot tube has a first opening, a second opening and two channels; the first opening is directed along a direction of flow in the tube and the second opening is directed opposite to the direction of flow in the tube; each channel pneumatically connects the openings to the differential pressure sensor.

25. The system of claim 23, wherein the tube further comprises a port for a hose and the sensor is a pressure sensor that is pneumatically connected to the port by the hose so that the pressure sensor can measure pressure in the port.

26. The system of claim 25, wherein the pressure sensor is a differential pressure sensor having a first port and a second port, the first port is pneumatically connected to the port for the hose and the second port is pneumatically connected to an interior of the tube.

27. The system of claim 25, wherein the pressure sensor is a differential pressure sensor having a first port and a second port, the first port is pneumatically connected to the port for the hose and the second port is pneumatically connected to an exterior of the tube.

28. The system of claim 25, wherein the port for the hose is connected to an interior of the tube via a capillary opening.

29. The system of claim 23, wherein the tube has an orifice plate mounted across an interior of the tube; the orifice plate being made of porous material; the tube has a first opening on a side upstream of the orifice plate and a second opening on a side downstream of the orifice plate in relative to a direction of flow;
wherein the sensor is a differential pressure sensor having a first port pneumatically connected to the first opening of the tube and a second port pneumatically connected to the second opening of the tube.

30. A method of producing the system of claim 23, the method comprising the steps of:
using a mask set, a tool set, an injection mold and/or a control program component to produce the tube;
using the mask set, the injection mold and/or the control program component to produce a sensor block that is adapted to be removably mounted on the tube.

31. A tube, comprising:
a wall and a first opening on the wall; and
a sterile filter.

32. The tube of claim 31 further comprising:
a longitudinal groove, extending parallel to a longitudinal axis of the tube, for releasable attachment of a sensor block.

33. A sensor system, comprising:
the tube of claim 32 and the sensor block;
wherein the sensor block further comprises a sensor; and a housing with a resilient clamp part for the sensor block to be removably mounted on the tube;
wherein the resilient clamp part is shaped such that the resilient clamp part can be plugged into the longitudinal grove of the tube along the radial direction and the resilient clamp part radially covers part of a wall of the tube

34. The tube of claim 31 further comprising:
a second opening on the wall;
a double pitot tube, the double pitot tube having a first opening, a second opening, a first channel, and a second channel; wherein the first channel pneumatically connects the first opening of the double pitot tube and the first opening of the tube on the wall; wherein the second channel pneumatically connects the second opening of the double pitot tube with the second opening of the tube on the wall; and
wherein the second opening of the tube on the wall is closed by a second sterile filter.

35. The tube of claim 31 further comprising:
an orifice plate mounted across an interior of the tube; the orifice plate being made of porous material and is located downstream of the first opening in relative to a direction of flow;
a second opening on the wall; the second opening located downstream of the orifice plate and is closed by a second sterile filter.

36. A method of producing the sensor system of claim 33, the method comprising the steps of:
using a mask set, a tool set, an injection mold and/or a control program component to produce the sensor block;
using the mask set, the injection mold, and/or the control program component to produce the tube.
